(19) United States
(12) Patent Application Publication
(10) Pub. No.: US 2021/0205199 A1
(43) Pub. Date: Jul. 8, 2021

(54) Dermatological Cosmetic Composition Comprising an SDKP Peptide or an Analog Thereof

(71) Applicant: RPM Dermatologie, Villeneuve les Avignon (FR)

(72) Inventor: Nicolas Riviere, Villeneuve les Avignon (FR)

(73) Assignee: RPM Dermatologie, Villeneuve les Avignon (FR)

(21) Appl. No.: 15/998,943

(22) PCT Filed: Feb. 20, 2017
(86) PCT No.: PCT/FR2017/050373

§ 371 Date: Aug. 17, 2018

(30) Foreign Application Priority Data
Feb. 18, 2016 (FR) 16 51328

(51) Int. Cl. A61K 8/64; A61Q 19/00; A61K 8/06

(52) U.S. Cl. CPC A61K 8/64; A61Q 19/00; A61K 2800/413; A61K 2800/57; A61K 8/06

(57) ABSTRACT

The invention relates to a lipid nanoparticle comprising an SDKP peptide conjugate or a biological peptide analog thereof, a method for obtaining said lipid nanoparticle, and the cosmetic use thereof as an anti-wrinkle agent or skin restructuring agent.

### INTRODUCTION

The subject of the present invention relates to a “neogoutte” (i.e. lipid nanoparticle) comprising an SDKP peptide conjugate or a biological peptide analog thereof, to a method for obtaining this neogoutte , and also to the cosmetic, preferably topical, use thereof as an anti-wrinkle agent or skin restructuring agent. More particularly, the subject of the present invention relates to an SDKP peptide conjugate or a biological peptide analog thereof, for such a use.

Skin aging is a complex phenomenon with various origins, which may be external (exposure to sun, cold, oxidizing agents, etc.) or intrinsic (genetic, protein-based, etc.).

The skin is a tissue, consisting of layers (epidermis, dermis and hypodermis, dermis and epidermis being linked by the dermal-epidermal junction (“DEJ”), having the role of a protective barrier between the internal and external environment.

The skin aging results at the surface in the appearance of wrinkles and fine lines. This is partly due to a relaxing of the cutaneous and subcutaneous tissues and to a loss of elasticity of the latter. Thus, the dermis provides the fundamental function of cohesion of the skin and constitutes a preferred target for anti-wrinkle treatment.

The loss of elasticity of the dermis/epidermis is explained by the conjuncture of several phenomena. It is partly due to the decrease in the amount of collagen and the reduction in the fibronectin content in the dermis. Furthermore, fibroblasts turn into fibrocytes or disappear. To this, is added a flattening of the DEJ which thus gradually loses its characteristic undulations, consequently decreasing the epidermis-dermis interface. The molecular networks of the skin are destructuring: the protein skeleton weakens, the basal keratinocytes show less adhesion, which results in an impairment of the biological functions and support of the DEJ.

Various means have been proposed against skin aging, ranging from solar protection to reinforcement of the DEJ through the activation of cell regeneration.

The Ac-SDKP-OH molecule is a natural tetrapeptide (acetyl-serine-aspartic acid-lysine-proline-OH) identified for its cosmetic properties at the level of the skin (EP 1 786 386, Bakala et al.).

However, this molecule hydrolyzes very easily, which makes its commercial exploitation difficult, in particular for cosmetic compositions of common use (i.e. stored at ambient temperature, opened and closed at the patient's convenience). The molecule is also very sensitive to enzymatic hydrolysis. The same is true for its analogs covered by EP 1 786 386. Thus, by virtue of its fragility, access to the deepest layers of the skin by this molecule is limited, thus restricting the effect of the treatment. Indeed, as explained above, skin aging is partly due to modifications of its deeper layers (dermal layers in particular). One solution considered for this has been to seek to limit the enzymatic hydrolysis by adding groups to the molecule. Effectively, in general, the more one “hindrance” around the peptide, the less the hydrolysis is facilitated by the enzyme. Nevertheless, in this case, when AcSDKP was substituted, in the laboratory, an ELISA assay kit no longer worked, implying that the molecule had at least partially lost its activity. Thus, the uncontrolled substitution of the AcSDKP peptide does not make it possible to overcome the technical problem linked to its fragility, even if the bare product is active on the surface of the skin.

Thus, it has been considered to use an emulsion/nanoemulsion formulation type in order to overcome these difficulties. Indeed, it is known that the lipid phase of an emulsion allows improved penetration of active ingredients into the skin.

Patent document FR 2 934 955 discloses the encapsulation of lipophilic or amphiphilic therapeutic agents in nanoemulsions. FR 2 934 955 discloses nanocapsules functionalized by a cyclopeptide (cRGD) coupled to a grafting cosurfactant, distearoylphosphatidylethanolamine poly(ethylene glycol) 5000 maleimide, thereby showing that this type of nanoemulsion is entirely compatible with peptides. However, since the peptide fragment is hydrophilic, it goes into the aqueous phase of the emulsion, and in this phase it is susceptible to undergoing hydrolysis, which hydrolysis is absolutely undesired.

Patent document FR 2 991 196 discloses targeting nanoparticles comprising a core consisting of a lipid phase (L1) or an aqueous phase (A1); at least one surfactant comprising a hydrophilic portion and a lipophilic portion; an internal membrane surrounding said core; an external membrane surrounding said internal membrane; and at least one targeting ligand comprising a lipophilic portion and a hydrophilic portion.

Surprisingly, the applicant has noticed that the stability and the profile of action of the “AcSDKP” molecule can be improved if the peptide is modified so as to be incorporated into systems as described in FR 2 934 955 or else FR 2 991 196 by replacing the N-terminal acetyl with a fatty chain, or by adding a C-terminal fatty chain. What is particularly surprising in the present invention is that the applicant has used this technology, in particular that of patent FR 2 991 196, not to bury the peptide in the oily phase, where the enzyme(s) cannot get to it, but in the aqueous phase, which is accessible. In particular, in the use of nanoparticles according to FR 2 991 196, instead of encapsulating the SDKP peptide in the core of the nanoparticle (which may be an aqueous phase), the applicant has formed a nanoparticle with a lipid core, and has grafted a fatty chain to SDKP in order to incorporate it into the nanoparticle in such a way that SDKP is placed toward the outside of the nanoparticle. The applicant then noticed that, when treating skin samples, surprisingly, the dermal layer of the skin was reached, whereas the peptide should have been degraded before reaching this layer. The activity of SDKP on the skin was significantly increased when SDKP is placed toward the outside of a neogoutte with a lipid core comprising at least one surfactant; an internal membrane surrounding said core; and an external membrane surrounding said internal membrane. Thus, the applicant does not yet understand very well the reason for this activity. The activity (and/or the absence of fragility) is probably at least partially linked to the size of the fatty chain grafted onto the SDKP peptide strand and/or the original arrangement of the peptide in the external aqueous phase of the neogoutte. In this regard, the teaching disclosed in FR 2934955 would be entirely usable with the novel grafted SDKP molecules of the present invention.

### SUMMARY OF THE INVENTION

The subject of the present invention relates to a peptide conjugate, which is preferentially amphiphilic and preferentially comprises less than 20 amino acids, comprising a peptide strand of formula SDKP, or a biological peptide analog thereof, characterized in that at least one linear or branched, saturated or unsaturated C₁₃-C₅₀, preferentially C₁₄-C₄₀, C₂₅-C₃₀, or else C₂₀-C₂₅, fatty chain is grafted at the N- and/or C-terminal end of the peptide strand, for example by means of a C═O group and/or an oxygen atom.

The subject of the present invention also relates to a neogoutte comprising:
a core consisting of a lipid phase (L₁);
at least one surfactant comprising a hydrophilic portion and a lipophilic portion;
an internal membrane surrounding said core, consisting of a lipid phase (L₂), comprising the lipophilic portion of said surfactant;
an external membrane, surrounding said internal membrane, consisting of an aqueous phase (A₁) comprising the hydrophilic portion of said surfactant; and
at least one peptide conjugate comprising a peptide strand of formula SDKP, or a biological peptide analog thereof, characterized in that at least one linear or branched, saturated or unsaturated, C₃-C₅₀, preferentially C₄-C₄₀, C₅-C₃₀, or else C₆-C₂₅, fatty chain is grafted at the N- and/or C-terminal end of the peptide strand, for example by means of a C═O group and/or an oxygen atom and said peptide conjugate is such that its lipophilic portion is in the lipid phase L₂ and its hydrophilic portion is in the phase A₁.

Furthermore, a subject of the present invention is a method for producing a neogoutte as defined above, characterized in that said method comprises the following steps:
a. preparing a lipid phase and an aqueous phase, at least one of the two phases comprising a surfactant, at least one of the two phases comprising the peptide stand of formula SDKP, or a biological peptide analog thereof, grafted with a fatty chain as presently defined;
b. emulsifying said lipid phase and said aqueous phase, resulting in the formation of neogouttes, optionally under pressure;
c. recovering the neogouttes formed.

Thus, the subject of the present invention relates to a cosmetic formulation comprising one or more neogouttes as presently defined, said formulation preferentially comprising between 1% and 25% by weight of neogouttes according to the present invention.

In addition, the present invention relates to a nanoemulsion comprising:
at least one dispersed lipid phase (L₃), and
at least one continuous aqueous phase (A₂) in which the aqueous phase comprises at least one surfactant,
characterized in that, at the interface between the aqueous phase (A₂) and the lipid phase (L₁), there is at least one peptide conjugate according to the present invention and/or in that said nanoemulsion comprises at least one neogoutte according to the present invention wherein optionally the lipids of (L₁) and (L₃), or (L₂) and (L₃) are identical.

The subject of the present invention also relates to a cosmetic use, preferably by topical application, of a peptide conjugate, of a neogoutte, of a nanoemulsion or of a cosmetic formulation according to the present invention, for restructuring or preserving the appearance of the skin, especially in depth at the dermal level.

Preferably, the nanoemulsion or the cosmetic formulation according to the present invention can contain up to 75% by weight of neogouttes according to the present invention, preferentially between 1% and 50% by weight of neogouttes according to the present invention, more preferentially between 2% and 25% by weight of neogouttes according to the present invention, even more preferentially between 3% and 15% by weight of neogouttes according to the present invention, and most preferably approximately 10% (i.e. between 9% and 11%) by weight of neogouttes according to the present invention.

More specifically, the subject of the present invention thus relates to a cosmetic use as defined above, characterized in that it is an anti-aging use.

### DEFINITIONS

### “Peptide Conjugate/Peptide Strand”

A “peptide strand” is a polymer of amino acids, which amino acids are linked to one another by at least one peptide or pseudopeptide bond. A “peptide bond” links a carbonyl group CO and an amino group NH in a peptide. The peptide bond is therefore an amide bond linking two amino acids together. Thus, the amino acids are termed “condensed” since they have lost at least one water molecule so as to form the peptide bond(s) in which they are involved. A “peptide conjugate” is a peptide strand conjugated, i.e. bonded, to a molecular fragment of another nature, for example a C₁₂ or C₁₆ hydrocarbon-based chain. It is accepted in the art that, in a peptide comprising a number X of amino acids, this in reality involves a number X of condensed amino acids, i.e. which have each lost a water molecule (if they are linked to one another by a peptide bond, typically).

### “Pseudopeptide Bond”

Thus, a peptide generally contains between 2 and approximately eighty amino acids, or even more, the upper limit not being clearly defined. A peptide comprising two amino acid residues is a dipeptide; a peptide comprising three amino acid residues is a tripeptide; a peptide comprising four amino acids is a tetrapeptide; and so on.

The expression “amino acid” comprises any molecule having at least one carboxylic acid, at least one amine and at least one carbon linking the amine and the carboxylic acid. Amino acids that can be used in the context of the present invention are all types of amino acids, whether they are natural or not, and in particular those found in the “SDKP” sequence. Thus, the amino acids that will more commonly be found in the context of the present invention will be chosen from serine (“Ser”, “S”), aspartic acid (“Asp”, “D”), lysine (“Lys”, “K”), proline (“Pro”, “P”), derivatives thereof such as enantiomers thereof, diastereoisomers thereof, positional isomers thereof, and “protected” forms thereof.

The expression “protected” forms of amino acids is understood to mean that the functional groups of these amino acids are substituted or hidden with appropriate “protective” groups chosen from those mentioned in the reference books by Greene “Protective Groups in Organic Synthesis”, Wiley, New York, 2007 4^{th} edition and/or Harrison et al. “Compendium of Synthetic Organic Methods”, Vol. 1 to 8 (J. Wiley & sons, 1971 to 1996).

The term “positional isomer” is understood to mean that some of the amino acids such as alanine can exhibit structural variations well known to those skilled in the art. For example, alanine can of course be in its alpha “a-Ala” form, but also its beta “(beta)-Ala”/ “β-Ala” form. With the exception of the case of alanine which comprises, in the context of the present invention, both the alpha form and the beta form, the alpha forms of natural amino acids are however preferred.

In the context of the present invention, the “natural amino acids” are Lys, Ala, Gin and Hys. Glycine can be considered to be a natural amino acid, but it does not comprise an asymmetrical carbon.

The term “enantiomers” (or “diastereoisomers”) is understood to mean that the asymmetrical carbons of the peptide backbone are of R or S configuration thus defining L or D (condensed) amino acids.

In addition, for reasons of production costs linked to the enantiomeric or diastereomeric purity of the amino acids used in the peptide synthesis, it may be advantageous for example to synthesize the peptides according to the present invention with one or more mixtures of amino acids of D and L forms. The result of the synthesis is thus a diastereomeric mixture of peptides according to the present invention. A subject of the present invention thus relates to a diastereomeric mixture of peptides having at least one D/L condensed amino acid in any proportions. Advantageously, the condensed amino acid(s) is (are) racemic.

The term “racemic mixture” is understood to mean that the enantiomer mixture is approximately 50/50% by weight of each of the enantiomers.

The term “approximately” comprises variations of±10% of each of the enantiomers in the mixture. Thus, a “racemic mixture” covers a 40/60% by weight mixture of a mixture of two enantiomers: N.B.: Strictly speaking, the expression “D/L mixture” relates to amino acids before they have been incorporated into the peptides. However, experimentally, the use of a racemic mixture of an amino acid in the synthesis of a small peptide, as is the case according to the present invention, makes it possible to obtain a mixture of approximately 50/50% of diastereoisomers of the peptide synthesized. Thus, through misuse of language, it is common in the field to refer to a “racemic mixture” of a condensed amino acid in a given peptide. It is obvious that, in this case, it is a mixture of diastereoisomers as explained above.

Advantageously, the proportions of amino acids used for the synthesis of the peptides according to the present invention are greater than or equal to 80%/20% by weight of each of the enantiomers relative to the total amount of the amino acid (i.e. L and D) in question.

In the context of the present invention, in order to consider that an amino acid is L or D, it must have a proportion greater than or equal to 90%/10% by weight of each of the enantiomers relative to the total amount of the amino acid (i.e. L and D) in question.

Preferentially, the “natural” amino acids of the peptides of formula (I) are of L form.

Peptides which are relatively restricted in terms of number of amino acids, such as those according to the present invention, are relatively easy to synthesize in liquid phase and/or on a solid support.

This is an undeniable advantage of the present invention.

The term “liquid-phase synthesis” is understood to mean the liquid-phase organic chemistry techniques taking all scales of magnitude/volume/weight together (i.e. techniques suitable for the research laboratory scale—for example a milligram, up to industrial amounts—for example a tonne).

The term “synthesis on a solid support” is understood to mean the organic chemistry techniques on a solid support, and in particular the techniques suitable for peptide synthesis on a solid support.

For example, peptide synthesis techniques are described in the handbook by Paul Lloyd-Williams, Fernando Albericio, Ernest Giralt, “Chemical Approaches to the Synthesis of Peptides and Proteins”, CRC Press, 1997 or Houben-Weyl, “Methods of Organic Chemistry, Synthesis of Peptides and Peptidomimetics”, Vol E 22a, Vol E 22b, Vol E 22c, Vol E 22d., M. Goodmann Ed., Georg Thieme Verlag, 2002.

Typically, these syntheses (both in liquid-phase and on a solid support) involve a “coupling-deprotection” strategy. This strategy consists in activating the C-terminal (i.e. COOH) end of an amino acid/peptide strand, and in bringing the latter into contact with another amino acid or peptide strand, the N-terminal (i.e. NH₂) end of which is free. The activation is carried out by conventional coupling techniques using DCC, BOP, PyBOP, anhydrides, mixed anhydrides, acid chlorides, etc. These coupling techniques can be used as variants for introducing fatty chains linked to the remainder of the molecule by means of a CO group or oxygen.

The amino acids or peptide strands involved in the reaction are suitably protected in order to allow this coupling selectively between the desired two N- and C-terminal ends. The functions that can react are of course hidden or bonded to suitable protective groups, as described above.

The “coupling-deprotection” step is repeated as many times as necessary with the desired amino acids/peptide strands in order to obtain the desired peptide. The final peptide, if it is supported by a resin, is detached. The protective groups are then removed. The final two steps can be carried out at the same time in the case of synthesis on a solid support. Such strategies are well known in the prior art. For example, the protection of the amino group of the amino acid can be carried out using a tert-butyloxycarbonyl group (hereinafter denoted Boc-) or a 9-fluorenylmethyloxycarbonyl (hereinafter denoted Fmoc-) group represented by the formula:

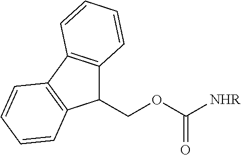

N.B.: “NHR” above representing the amine grafted with Fmoc.

The protection is carried out according to the known methods of the prior art. For example, protection with the Boc- group can be obtained by reacting the amino acid with di-tert-butyl pyrocarbonate (Boc₂O).

These techniques are very common in the field, so that mention is regularly made of “Boc or Fmoc strategies” for peptide synthesis.

For example, the peptides according to the present invention are synthesized by a series of deprotection/coupling cycles, preferentially by means of the Fmoc/tBu strategy.

The progress of each deprotection/coupling step is monitored by the usual techniques, such as mass spectrometry.

Advantageously, the free N-terminal end of the peptide which has not reacted in the coupling reaction is acetylated using an acetic anhydride solution (step termed “capping”).

The final step of deprotecting the side chains is preferentially carried out in an acid medium, more preferentially in the presence of trifluoroacetic acid (TFA).

Advantageously, the synthesis is carried out on a solid support, such as on Rink amide, CTR (“chlorotrityl” type), Wang, etc., resins, and the final acid deprotection step also makes it possible to detach (“cleave”) the peptide from the resin.

The usual techniques for purifying peptides, such as precipitation, crystallization, liquid chromatography (normal phase or preferentially reverse phase and at high pressure) are applied to the peptides obtained.

### “SDKP Peptide”

The term “SDKP” represents a peptide strand having the structure below:

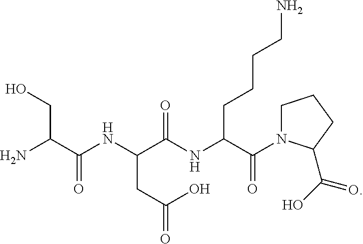 [SMILES: NCCCCC(NC(=O)C(CC(=O)O)NC(=O)C(N)CO)C(=O)N1CCCC1C(=O)O]

Advantageously, the amino acids are of L configuration:

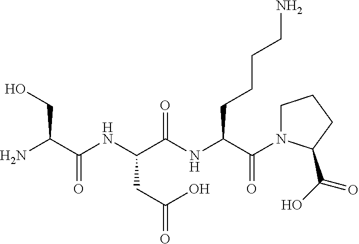 [SMILES: NCCCC[C@H](NC(=O)[C@H](CC(=O)O)NC(=O)[C@@H](N)CO)C(=O)N1CCC[C@H]1C(=O)O]

The functional groups (e.g. NH₂, OH, COOH) can be in protonated or deprotonated form depending on the surrounding pH. These groups can also be protected with conventional protective groups as found in the handbooks “Protective Groups in Organic Synthesis”, Wiley, New York, 2007 4^{th} edition and/or Harrison et al. “Compendium of Synthetic Organic Methods”, vol. 1 to 8 (J. Wiley & sons, 1971 to 1996). Thus typically, the COOH functions can be protected with an ester group such as OtBu, OMe, OEt, or else O-benzyl, the OH function can be protected with an ether of tBu, Me, Et or benzyl, and the NH₂ function can be protected with an acido-labile group such as Boc or a baso-labile group such as Fmoc.

### “Biological Analog”

In the context of the present invention, a “biological analog” is a compound having biological properties similar to the initial molecule in question, in this case Ac-SDKP-OH. Thus, in order to determine whether a compound is a biological analog of “Ac-SDKP-OH”, it is required to know whether the same physiological action mechanisms are generated. Conventionally, a comparative test (between the molecule of interest and “Ac-SDKP-OH) in vitro makes it possible to easily verify this. In the context of the present invention, a comparative test on human skin cells makes it possible to know whether the compound of interest is a biological analog of “Ac-SDKP-OH”. Preferably, the biological analog of “Ac-SDKP-OH” is an anti-skin-aging biological analog (anti-wrinkle or skin restructuring). Thus, the term “similar biological properties” is understood to mean, in the context of the present invention, that the molecule of interest has an activity at least equal to 90% of the activity of Ac-SDKP-OH tested under the same conditions, in particular on skin cells. Typically, the biological analogs according to the present invention will comprise the peptide strand of formula (II) below:

—Y₁—Y₂—Y₃—Y₄— (II)

wherein,
Y₁ represents the N-terminal end of the peptide fragment,
the fragments Y₃—Y₄ or —Y₄ possibly being absent from the peptide fragment of formula (II),
Y₁ represents serine or a structural analog of serine,
Y₂ represents aspartic acid or a structural analog of aspartic acid,
Y₃ represents lysine or a structural analog of lysine,
Y₄ represents proline or a structural analog of proline.

The term “structural analog of serine” can be understood to mean, in the context of the present invention, a serine functionalized with a C₁-C₅ alkyl, C₁-C₅ alkene (such as N-methylserine), or else molecules known to have properties close to serine, such as 4-amino-3-hydroxybutanoic acid, 2-amino-3-methoxybutanoic acid, or else O-benzyl phosphoserine.

The term “structural analog of aspartic acid” can be understood to mean, in the context of the present invention, an aspartic acid functionalized with a C₁-C₅ alkyl, a C₁-C₅ alkene (such as N-methylaspartic acid), or else molecules known to have properties close to aspartic acid, such as glutamic acid, 2,6-diaminoheptanedioic acid, or else the benzoate of the aspartic acid side chain (NH₂-Asp(OBn)-OH).

The term “structural analog of lysine” can be understood to mean, in the context of the present invention, a lysine functionalized with a C₁-C₅ alkyl, a C₁-C₅ alkene (such as N-methyllysine, doubly methylated lysine (NH₂-Lys(Me₂)-OH)), or else molecules known to have properties close to lysine, such as arginine, lysine azide (NH₂-Lys(N₃)-OH), ornithine, 2-amino-3-guanidinopropionic acid, citrulline, or else acetylated lysine (NH₂-Lys(Ac)-OH).

The term “structural analog of proline” can be understood to mean, in the context of the present invention, a proline functionalized with a C₁-C₅ alkyl, a C₁-C₅ alkene (such as N-methylproline), or else molecules known to have properties close to proline, such as trans-4-fluoroproline, trans-4-tert-butoxyproline, 3-phenylpyrrolidine-2-carboxylic acid, (2S,3aS,7aS)-octahydro-1H-indole-2-carboxylic acid, 4-benzylpyrrolidine-2-carboxylic acid, 4-phenylpyrrolidine-2-carboxylic acid, or else 4-hydroxypyrrolidine-2-carboxylic acid.

In the context of the present invention, the biological analog is a biological peptide analog advantageously having “SD”, “SDK” or “SDKP” in its sequence. Preferably, the “SD”, “SDK” or “SDKP” strand is included in the last 10 amino acids of the N-terminal end of the biological peptide analog. More preferably, the “SD”, “SDK” or “SDKP” strand is included in the last 5 amino acids of the N-terminal end of the biological peptide analog. Even more preferably, the “SD”, “SDK” or “SDKP” strand is at the N-terminal end of the biological peptide analog.

Advantageously, the biological peptide analog comprises less than 20 amino acids, more advantageously less than 10 amino acids, and even more advantageously it comprises less than 6 amino acids, or less than 5 amino acids. The Ac-SDKP peptide analogs are well known in the art and are illustrated for example in the articles Ma X et al. (Int J Cardiol. 2014 Aug 1;175(2):376-8), Zhuo J L et al (Am J Physiol Heart Circ Physiol. 2007 Feb;292(2), Gaudron S. et al. (Stem Cells, 1999,17, 2,100-106; J. Med. Chem., 1997,40, 24, 3963-3968) or in international patent application WO 2004/096292 (and the references which are mentioned in this application).

For example, Ac-SDKP-OH analogs identified in the prior art are: N-acetyl-Ser(CH₂-NH)-Asp-Lys-Pro-OH; N-acetyl-Ser-Asp(CH₂-NH)-Lys-Pro-OH; N-acetyl-Ser-Asp-Lys(CH₂-NH)-Pro-OH; N-acetyl-Ser-Asp-Lys-Pro-NH₂; N-acetyl-Ser-Asp-Lys-OH. It is implicit in the context of the present invention that the “Ac” fragment in these molecules can be replaced with a fatty chain.

### “Fatty Chain”

According to the present invention, the term “fatty chain” is understood to mean any linear or branched chain which is hydrophobic organic or (partially) inorganic in nature, preferentially comprising at least 3 atoms (when this value is not specified) successively bonded to one another. This fatty chain can be exclusively hydrocarbon-based, or can comprise heteroatoms such as O, S, Si, N, F, CI, Br, I or P.

Preferably, the fatty chains according to the present invention are exclusively hydrocarbon-based chains, optionally unsaturated, which can comprise one or more aromatic nuclei (i.e. phenyl). Thus, the fatty chain may be linear or branched, saturated or unsaturated, C₁₃-C₅₀.

### “Substituted or Unsubstituted, Linear or Branched C₁₃-C₅₀ Alkyl”

The expression “substituted or unsubstituted, linear or branched C₁₃-C₅₀ alkyl” represents a linear or branched, saturated hydrocarbon-based chain comprising from 13 to 50 carbon atoms, such as for example an n-tridecyl (C13), n-tetradecyl (C14), n-pentadecyl (C15), n-hexadecyl (16), n-heptadecyl (C17), n-octadecyl (C18), n-nonadecyl (C19), n-eicosyl (C20), 1-methyldodecyl (C13), 2-methyldodecyl (C13), 1-ethylundecyl (C13), 2-ethylundecyl (C13), etc., group. The alkyls may be substituted or unsubstituted. Preferentially, the alkyls may be substituted with one or more halogens such as F, Cl, Br, I, or with other fragments, such as OH, SH, NO₂, NH₂, COOH. The alkyls may also comprise or consist of a cyclic portion, such as a cyclohexyl or cyclopentyl unit.

### “Aryl”

The term “aryl” group is understood to mean an aromatic group preferably comprising from 5 to 18 carbon atoms, comprising one or more rings and optionally comprising one or more heteroatom(s), in particular an oxygen, a nitrogen or a sulfur, such as for example a phenyl, furan, indole, pyridine, naphthalene, anthracene, etc., group. Preferably, the term “aryl” group is understood to mean the phenyl group.

### “Substituted or unsubstituted, linear or branched C₂₁-C₅₀ alkylaryl”

In the present invention, the expression “substituted or unsubstituted, linear or branched C₂₁-C₅₀ alkylaryl” represents a C₁ to C45 alkyl fragment, as defined above, in which at least one hydrogen atom has been replaced with at least one aryl group, in which the total sum of the carbon atoms present in the alkylaryl is between 21 and 50. The alkylaryls according to the present invention may be substituted or unsubstituted. Preferentially, the alkylaryls according to the present invention may be substituted with one or more halogens such as F, CI, Br or I, or with other fragments, such as OH, SH, NO₂, NH₂ or COOH.

For example, the C₁ to C₄₅ alkyls substituted with one or more aryls comprise the 1-phenylheptyl, 2-phenylheptyl, 3-phenylheptyl, 4-phenylheptyl, 5-phenylheptyl, 6-phenylheptyl, 7-phenylheptyl, 1-phenyloctyl, 2-phenyloctyl, 3-phenyloctyl, 4-phenyloctyl, 5-phenyloctyl, 6-phenyloctyl, 7-phenyloctyl, 8-phenyloctyl, 1-phenylnonyl, 2-phenylnonyl, etc., 1-naphthylpropyl, 2-naphthyl-propyl, 3-naphthylpropyl, 1-anthracylmethyl, etc., groups.

Preferably, the C₁ to C₄₅ alkyls substituted with one or more aryls are C₇-C₄₄ alkyls substituted with at least one phenyl. Even more preferably, the C₁ to C₄₅ alkyls substituted with one or more aryls are C₁₂-C₄₄ alkyls substituted with at least one phenyl.

### “Substituted or Unsubstituted, Linear or Branched C₁₃-C₅₀ Alkenyl”

In the present invention, the expression “C₁₃-C₅₀ substituted or unsubstituted, linear or branched alkenyl” represents a hydrocarbon-based fragment comprising at least one unsaturation, i.e. covalent double bond. The alkenyls according to the present invention may or may not be substituted. Preferentially, the alkenyls may be substituted with one or more halogens such as F, CI, Br or I, or with other fragments, such as OH, SH, NO₂, NH₂, COOH.

Examples of such alkenyls are an n-1-tridecenyl (C13), n-2-tridecenyl (C13), n-3-tridecenyl (C13), n-4-tridecenyl (C13), n-5-tridecenyl (C13), n-6-tridecenyl (C13), n-7-tridecenyl (C13), n-8-tridecenyl (C13), n-9-tridecenyl (C13), n-10-tridecenyl (C13), n-11-tridecenyl (C13), n-12-tridecenyl (C13), n-1-tetradecenyl (C14), n-1-pentadecenyl (C15), n-1-hexadecenyl (16), n-1-heptadecenyl (C17), n-1-octadecenyl (C18), n-1-nonadecenyl (C19), n-1-eicosenyl (C20), etc., group.

The alkenyls may be substituted or unsubstituted. Preferentially, the alkenyls may be substituted with one or more halogens such as F, Cl, Br, I, or with other fragments, such as OH, SH, NO₂, NH₂, COOH.

### “Guanidine” or “Guanidinium”

A guanidine group is represented by the formula:

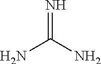

This group is typically linked to another molecule by one of the nitrogen atoms in the representation above. The protonation of this guanidine forms a cation which is stabilized by resonance. The positive charge in this case allows this group to have a lipophilicity. This is typically the case with protonated arginines for example, used in order to increase the lipophilicity of a peptide strand.

### “Pharmaceutically Acceptable Salts”

According to the present invention, the expression “pharmaceutically acceptable salts” relates to an acid addition salt preferably formed by pharmaceutically acceptable free acids. An acid addition salt can be obtained from inorganic acid(s) such as hydrochloric acid, nitric acid, phosphoric acid, sulfuric add, hydrobromic acid, hydriodic acid, nitrous add acid phosphorus acid, or from non-toxic organic acid(s) such as a mono/dicarboxylate aliphatic compound, a substituted phenyl alkanoate group, a hydroxyalkanoate group, an alkandloate group, aromatic acids and aliphatic/aromatic sulfonic acids.

The pharmaceutically acceptable non-toxic salts may be a sulfate, a pyrosulfate, a bisulfate, a sulfite, a bisulfite, a nitrate, a phosphate, a monohydrogen phosphate, a dihydrogen phosphate, a metaphosphate, a pyrophosphate, a chloride, a bromide, an iodide, a fluoride, an acetate, a propionate, a decanoate, a caprylate, an acrylate, a formiate, an isobutylate, a caprate, a heptanoate, a propiolate, an oxalate, a malonate, a succinate, a fumarate, a benzoate, a chlorobenzoate, a methylbenzoate, a dinitrobenzoate, a hydroxybenzoate, a methoxybenzoate, a phthalate, a terephthalate, a benzenesulfonate, a toluenesulfonate, a chlorobenzene, xylenesulfonate, a phenylacetate, a phenylpropionate, a phenylbutylate, a citrate, a lactate, a hydroxybutylate, a glycolate, a malate, a tartrate, a methanesulfonate, a propanesulfonate, a 1-naphthalenesulfonate, a naphthalene2-sulfonate and a mandelate.

The acid addition salts of the present invention can be prepared by the conventional methods known to those skilled in the art, For example, the compounds of the present invention are dissolved in an acidic aqueous solution. Next, the salt is obtained by precipitation from the solution using a water-miscible organic solvent such as methanol, ethanol, acetone or acetonitrile. The salts can be obtained by any other known means.

Pharmaceutically acceptable metal salts can be prepared using one or more bases. The alkali metal or the alkali metal salt can be obtained by means of the following methods: dissolution of the compound in an excess of alkali metal hydroxide or in a solution of alkaline-earth metal hydroxide; filtration of the insoluble compound of the salt; evaporation of the solution and drying thereof. At this time, the metal salt is preferably prepared in a pharmaceutically suitable form, such as a sodium salt, a potassium salt or a calcium salt.

### “Neogoutte”/“Lipid Nanoparticle”

In the context of the present invention, the active peptides can be inserted into a nanoparticle as disclosed in FR 2 991 196. This nanoparticle comprises several lipid/aqueous phases. By virtue of its particular properties found with the peptides of the invention, the applicant has assigned the name “neogoutte” to these nanoparticles. Nevertheless, according to the present invention, the term “neogoutte” is a synonym for “lipid nanoparticle” and these expressions are therefore equivalent.

### “Lipid Phase”

According to the present invention, the term “lipid phase” is understood to mean any phase having affinity with organic solvents and lipids (oils or waxes) and avoiding being in contact with a polar solvent, such as water. It may be a question of the fatty substances as defined in FR 2 934 955 and FR 2 991 196. For example, the lipid phase may comprise phospholipids as defined on page 7, lines 7 to 13 of FR 2 934 955, lecithin as defined on page 7, lines 14 to 19 of FR 2 934 955, fatty acids as defined on page 7, lines 20 to 25 of FR 2 934 955, the amphiphilic lipids as disclosed on page 13, line 8 to page 14, line 30 of FR 2 991 196.

### “Surfactant”

In the context of the present invention, the expression “surfactant” represents an amphiphilic molecule having two parts of different polarities, one being lipophilic (nonpolar) and the other being hydrophilic (polar). The surfactant may be ionic, non-ionic or zwitterionic (cationic and anionic). For example, the surfactants according to the present invention can be chosen from those disclosed in FR 2 991 196 and/or FR 2 934 955, such as those found on page 4, line 28 to page 6, line 3 of FR 2 991 196, or page 11, line 19 to page 12, line 11 of FR 2 934 955.

### “Topical Route”

The term “topical route” is understood to mean the administration of the peptides and/or of the compositions containing the peptides directly to the areas of skin to be treated.

### “Anti-Aging Use”

The objective of the subject of the present invention is in particular an anti-aging use, i.e. use against intrinsic skin aging.

The term “intrinsic skin aging” is understood to mean that it is a natural modification of the skin as a function of time, which is characterized by the appearance of wrinkles, fine lines, crevices, etc.

The subject of the present invention also relates to an anti-aging kit. The term “anti-aging kit” is understood to mean a device comprising a set of elements, at least one element of which is intended to repair the intrinsic skin aging and at least one element of which comprises or consists of a peptide according to the invention. Preferentially, said peptide is included in a composition according to the invention.

### DETAILED DESCRIPTION

The subject of the present invention relates more particularly to a peptide conjugate as defined above, characterized in that said conjugate is of formula (I) below:

R₁—X₁—X₂—X₃—X₄—A₁—R₂ (I)

wherein:
R_{1,} which is on the N-terminal side, is a hydrogen atom, or a fatty chain chosen from a substituted or unsubstituted, linear or branched C₁₃-C₅₀ alkyl group, a substituted or unsubstituted, linear or branched C₂₁-C₅₀ alkylaryl group, a substituted or unsubstituted, linear or branched C₁₃-C₅₀ alkenyl group, or an R₃—CO—, R₃—COO— or R₃-COO- group wherein R₃ is a substituted or unsubstituted, linear or branched C₁₃-C₄₉ alkyl or alkenyl group, the substitutions including F, Cl, or any lipophilic heteroatom or heteroatom group such as a guanidine or a guanidinium,
X₁ is an (L)-serine condensate,
X₂ is an (L)- and/or (D)-aspartic acid or (L)- and/or (D)-glutamic acid condensate,
X₃ is an (L)- and/or (D)-lysine, (L)- and/or (D)-arginine or (L)- and/or (D)-ornithine condensate,
X₄ is an (L) and/or (D)-proline condensate,
X_{4,} or X₃ and X_{4,} being optionally absent,
the bonds linking X₁ to X_{2,} X₂ to X₃ where appropriate, and X₃ to X₄ where appropriate, possibly being peptide or pseudopeptide bonds,
A₁ is a covalent bond, an NH group or an oxygen atom,
R₂ is a hydrogen atom, or a fatty chain chosen from a substituted or unsubstituted, linear or branched C₁₃-C₅₀ alkyl group, a substituted or unsubstituted, linear or branched C₂₁-C₅₀ alkylaryl group, a substituted or unsubstituted, linear or branched C₁₃-C₅₀ alkenyl group, or an R₄—CO— or R₄—OCO— group in which R₄ is a substituted or unsubstituted, linear or branched C₁₃-C₅₀ alkyl or alkenyl group, the substitutions including F, Cl, or any lipophilic heteroatom or heteroatom group such as a guanidine or guanidinium group,
R₁ and R₂ not being able to both be hydrogen atoms,
or a pharmaceutically acceptable salt thereof.

The term “condensate” is understood to mean, in the context of the present invention, the remaining portion of the amino acid following the formation of the peptide bond between two amino acids (in the case of a reaction between two amino acids), one water molecule having been eliminated. For example, in the case of serine:

In the figure above, at each condensation step, the elimination of H₂O implies that OH or H is provided by the serine. Nevertheless, if the amino acid is not condensed on its two ends (N-terminal and C-terminal ends), through misuse of language it is considered to be a condensate. This explains why, in peptide chemistry, the nomenclature agrees to add an H at the N-terminal or an OH at the C-terminal, as appropriate: H-AA₁-AA₂- . . . AA₂-OH. All this is part of the general knowledge of those skilled in the art.

The subject of the present invention also relates to a neogoutte (lipid nanoparticle) according to the present invention, characterized in that the peptide conjugate or the biological analog thereof is as presently described.

The subject of the present invention also relates to a neogoutte (lipid nanoparticle) according to the present invention, characterized in that it also comprises the peptide conjugate of formula Ac-SDKP-OH, Ac-SDKP-NH₂ or a mixture of the two.

The subject of the present invention also relates to a neogoutte as defined above, characterized in that the peptide conjugate coupled to this neogoutte is of formula (I) as defined above, in which:
R₁, which is on the N-terminal side, is a hydrogen atom, a substituted or unsubstituted, linear or branched C₃-C₅₀ alkyl group, a substituted or unsubstituted, linear or branched C₈-C₅₀ alkylaryl group, a substituted or unsubstituted, linear or branched C₃-C₅₀ alkenyl group, or an R₃—CO—, R₃—OCO— or R₃—COO— group in which R₃ is a substituted or unsubstituted, linear or branched C₃-C₅₀ alkyl or alkenyl group, the substitutions including F, Cl, or any lipophilic heteroatom or heteroatom group such as a guanidine or guanidinium group, and
R₂ is a hydrogen atom, a substituted or unsubstituted, linear or branched C₃-C₅₀ alkyl group, a substituted or unsubstituted, linear or branched C₈-C₅₀ alkylaryl group, a substituted or unsubstituted, linear or branched C₃-C₅₀ alkenyl group, or an R₄—CO— or R₄—OCO— group in which R₄ is a substituted or unsubstituted, linear or branched C₃-C₅₀ alkyl or alkenyl group, the substitutions including F, Cl, or any lipophilic heteroatom or heteroatom group such as a guanidine or guanidinium group.

Preferably, the subject of the present invention also relates to a neogoutte as defined above, characterized in that R₁ is a substituted or unsubstituted, linear or branched C₆-C₃₀, preferentially C₁₀-C₂₀, alkyl group, or an R₃—CO— group in which R₃ is a substituted or unsubstituted, linear or branched C₅-C₃₀, preferentially C₉-C₂₀, alkyl or alkenyl group, these substitutions including F, Cl, or any lipophilic heteroatom or heteroatom group such as a guanidine or guanidinium group.

More preferably, the subject of the present invention relates to a neogoutte as defined above, characterized in that R₁ is a substituted or unsubstituted, linear or branched C₁₂-C₁₈, preferentially C₁₄-C₁₆, alkyl group, or an R₃—CO— group in which R₃ is a substituted or unsubstituted, linear or branched C₁₀-C₁₈, preferentially C₁₃-C₁₆, alkyl or alkenyl group, the substitutions including F, Cl, or any lipophilic heteroatom or heteroatom group such as a guanidine or guanidinium group.

Even more preferably, the subject of the present invention relates to a neogoutte as defined above, characterized in that R₁ is a substituted or unsubstituted, linear or branched C₁₂, C₁₃, C₁₄, C₁₅, C₁₆, C₁₇, C₁₈, C₁₉ or C₂₀ alkyl group or an R₃—CO— group in which R₃ is a substituted or unsubstituted, linear or branched C₁₂, C₁₃, C₁₄, C₁₅, C₁₆, C₁₇, C₁₈, C₁₉ or C₂₀ alkyl or alkenyl group, the substitutions including F, Cl, or any lipophilic heteroatom or heteroatom group such as a guanidine or guanidinium group.

In addition, the subject of the present invention relates to neogouttes as defined above, comprising a peptide conjugate according to the present invention, preferentially of formula (I), characterized in that the peptide is present at the surface of the neogoutte.

Typically, during the production of the neogouttes according to the present invention, the peptide conjugate (I) is distributed between the free aqueous phase and the superficial layer of said neogouttes. In other words, a portion of the peptide conjugate (I) attaches to the neogoutte by means of its fatty chain and the other portion of peptide conjugate (I) remains free in the aqueous phase. This distribution of the peptide conjugate according to the present invention varies according to the nature of the fatty chain on said peptide conjugate: the larger the fatty chain, naturally, the larger the proportion of peptide conjugate bonded to the neogoutte. This distribution of the peptide conjugate also varies, but to a lesser extent, according to the nature of the L₁ and/or L₂ phase. Thus, it is at least necessary for the fatty chain of the peptide conjugate (I) to comprise three carbon atoms in order to begin to observe neogouttes bonded with peptide conjugates (I); a peptide conjugate (I) comprising a fatty chain of from 10 to 15 carbon atoms will typically induce a rate of bonding to the neogouttes of between 20% and 50% by weight. For example, a peptide conjugate (I) comprising a fatty chain of 12 carbon atoms will typically induce a rate of bonding to the neogouttes of approximately 25% to 30% by weight under the conventional conditions according to the present invention. Thus, in general, the neogoutte as defined above is characterized in that it comprises between 5% and 95% by weight of peptide conjugate (I) bonded to the neogoutte, advantageously between 10% and 80% by weight, more advantageously between 20% and 60% by weight, even more advantageously between 25% and 50% by weight, with respect to the total amount of peptide conjugate (I).

Advantageously, the neogouttes according to the present invention are diluted in an aqueous solvent, preferentially comprising at least 50% of water by weight/total weight, more preferentially 75% of water, even more preferentially at least 90% of water, such as at least 95%, at least 97% or at least 99% of water. Preferably, this dilution is at least 1:1 by weight (weight of neogouttes: weight of aqueous solvent), preferentially at least 1:5 (weight of neogouttes:weight of aqueous solvent), more preferentially at least 1:7 (weight of neogouttes : weight of aqueous solvent). Most preferably, this dilution is between 1:5 by weight (weight of neogouttes : weight of aqueous solvent) and 1:100 (weight of neogouttes:weight of aqueous solvent), advantageously between 1:5 by weight (weight of neogouttes:weight of aqueous solvent) and 1:50 (weight of neogouttes:weight of aqueous solvent), such as approximately 1:10 (weight of neogouttes:weight of aqueous solvent).

Thus, the subject of the present invention relates to a neogoutte as defined above, characterized in that said neogoutte comprises at least one of the following constituents:
at least one C₅-C₃₀ fatty acid, which is optionally hydrogenated and/or in glycol ester form, in the (L₁) phase,
a C₁-C₃₀ fatty acid ester of polyoxyethylene (10-100) as surfactant,
at least one C₅-C₃₀ fatty acid, which is optionally hydrogenated and/or in glycol ester form, in the (L₂) phase, and/or
at least one preservative, for example of phenoxyethanol type, in the (A₁) phase.

Preferentially, the subject of the present invention relates to a neogoutte as defined above, characterized in that said neogoutte comprises at least one of the following constituents:
at least one C₁₀-C₂₀ fatty acid, which is optionally hydrogenated and/or in glycol ester form, in the (L₁) phase,
a C₁₀-C₂₀ fatty acid ester of polyoxyethylene (10-100) as surfactant,
at least one C₁₀-C₂₀ fatty acid, which is optionally hydrogenated and/or in glycol ester form, in the (L₂) phase, and/or
at least one preservative, for example of phenoxyethanol type, in the (A₁) phase.

More preferentially, the subject of the present invention relates to a neogoutte as defined above, characterized in that said neogoutte comprises at least one of the following constituents:
at least one C₁₂, C₁₃, C₁₄, C₁₅, C₁₆, C₁₇, C₁₈, C₁₉ or C₂₀ fatty acid, which is optionally hydrogenated and/or in glycol ester form, in the (L₁) phase,
a C₁₂, C₁₃, C₁₄, C₁₅, C₁₆, C₁₇, C₁₈, C₁₉ or C₂₀ fatty acid ester of polyoxyethylene (10-100) as surfactant,
at least one C₁₂, C₁₃, C₁₄, C₁₅, C₁₆, C₁₇, C₁₈, C₁₉ or C₂₀ fatty acid, which is optionally hydrogenated and/or in glycol ester form, in the (L₂) phase, and/or
at least one preservative, for example of phenoxyethanol type, in the (A₁) phase.

More specifically, the subject of the present invention relates to a neogoutte as defined above, characterized in that the peptide conjugate comprises the sequence (L)S-(L)D-(L)K-(L)P.

The subject of the present invention can relate to a neogoutte as defined above, characterized in that the peptide conjugate comprises the sequence (L)S-(L)D-(L)K.

The subject of the present invention relates to a neogoutte as defined above, characterized in that the peptide conjugate comprises the sequence (L)S-(L)D.

The subject of the present invention also relates to a method for producing the neogoutte according to the present invention. The method is close to that described in FR 2 991 196 (cf. page 17, line 26 to page 19, line 27), the only difference being that the peptide conjugate (active ingredient) according to the present invention is grafted onto the external part and not the core of the neogoutte. This can be carried out by adding the peptide conjugate during the formation of the neogoutte or afterwards. Preferably, by virtue of the affinities of the peptide conjugate according to the invention, said conjugate will naturally position itself at the surface of the neogoutte. However, it is also possible to add this peptide conjugate precisely at the time the internal membrane L₂ forms, by means of the techniques described in FR 2 991 196.

Thus, the subject of the present invention relates to a cosmetic composition comprising a peptide conjugate according to the present invention in a content of between 1×10⁻⁹% and 10% by weight relative to the total amount of said composition. Preferentially, the peptide conjugate according to the present invention is included in said cosmetic composition in a neogoutte according to the present invention. Advantageously, the cosmetic composition according to the present invention contains the peptide conjugate in a content of between 1×10⁻⁷% and 1% by weight, more advantageously in a content of between 1×10⁻⁶% and 0.1% by weight, or even in a content of between 2×10⁶% and 0.01% by weight. More preferably, the content of peptide conjugate according to the present invention is approximately 5×10⁻⁶% by weight.

One particular embodiment according to the present invention relates to a cosmetic composition comprising an SDKP analog as defined above, for example a peptide conjugate of formula (I), with, in addition, Ac-SDKP-OH and/or Ac-SDKP-NH₂. Preferably, Ac-SDKP-OH and/or Ac-SDKP-NH₂ is at least partially included in the neogoutte according to the present invention.

The cosmetic composition according to the present invention can be packaged in any means suitable for its application, such as a pot, a tube, a bottle, a spray or a box.

The cosmetic composition according to the present invention can be administered topically one or more times a day, preferentially at least once in the morning and once in the evening.

The cosmetic composition can be in the form of a cream, a gel, a lotion, a serum or else a paste.

### FIGURES

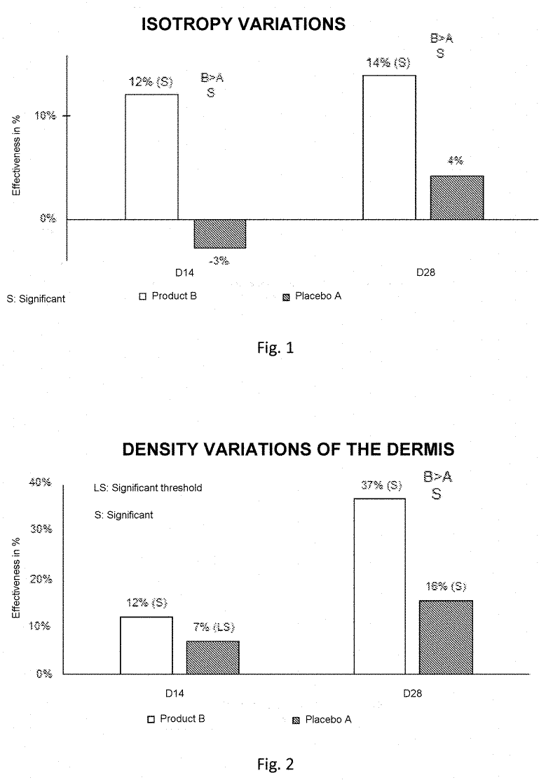

The histogram of FIG. 1 represents the variations in the isotropy following the comparative study according to example 4 below. The product B comprises lauroyl-SDKP-OH, whereas the placebo A is a neogoutte formulation free of SDKP analogs. The significant effect on the relief of the skin (isotropy—orientation of the lines of the skin) of the product B compared with the placebo can be seen.

The histogram of FIG. 2 represents the variations in the density of the dermis following the comparative study according to example 4 below. The product B comprises lauroyl-SDKP-OH, whereas the placebo A is a neogoutte formulation free of SDKP analogs. The significant effect on the density of the dermis of the product B compared with the placebo can be seen.

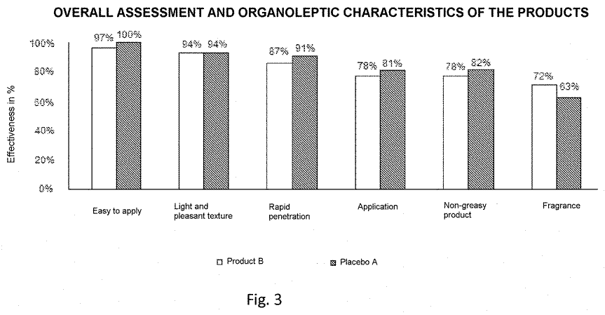

The histogram of FIG. 3 represents the overall assessment and organoleptic characteristics of the products (ease of application, light and pleasant texture, rapid penetration, application, non-greasy product, fragrance). It can be seen that the formulations tested have organoleptic characteristics and general assessments which are similar and acceptable.

### EXAMPLES

### Example 1: Synthesis of the “lauroyl-SDKP-OH” Compound

The synthesis described below is for illustration purposes. It can for example be carried out on a solid support (resin) by Fmoc strategy (on an automated device for amounts less than 50 mg and manually for larger amounts). The levels of charges of the resins and the concentrations of the reagents (coupling agents, bases, acids, scavengers) can vary according to the common practice in the art.

The starting materials used can be:
natural amino acids protected on their N-terminal ends (Fmoc protection) and on their side chains (acido-labile protective groups which are therefore compatible with the Fmoc strategy);
activators (HOBT/DIC). These products are eliminated by washing throughout the synthesis, then during the precipitation phase and, finally, during the purification phase;
washing solvents (DMF, DCM). These products are eliminated by washing throughout the synthesis, then during the precipitation phase and, finally, during the purification phase;
Wang resin precharged with the Fmoc-L-Pro amino acid. The initial, or reduced, level of proline charge of the resin can vary, but is typically between 0.1 and 2.0 mol/g of resin, preferentially between 0.5 and 1.5 mol/g, more preferentially approximately 0.75 mol/g ±10%. The resin is eliminated by simple filtration at the end of the synthesis;
the usual bases for removing Fmoc are used, such as triethylamine, DBU, piperidine, etc. Piperidine was in particular used here;
the usual acids for removing the peptide from the support were used (e.g. TFA, HCI, acetic acid, etc.). In the case of “Wang” resins, TFA (trifluoroacetic acid) with a scavenger, such as usual silicon derivatives in the art (e.g. TIS).

The synthesis is carried out in the following way:
Starting point: Resin precharged with the Fmoc-Proline;
1. Grafting of the second amino acid, lysine:
   Deprotection of the supported proline with piperidine (for example 20% in DMF) in order to remove the Fmoc group;
   3 series of washes with DMF;
   Coupling of Fmoc-L-Lys(Boc)-OH on the extending peptide, by using HOBt, DCI;
   3 series of washes with DMF;
   Acetylation using acetic anhydride or the equivalent acyl chloride in order to block any unreacted amines of Pro;
   3 series of washing with DMF;
2. Grafting of the aspartic acid Fmoc-L-Asp(OtBu)-OH:
   The same steps as for the grafting of lysine are reproduced for aspartic acid.
3. Grafting of the serine Fmoc-L-Ser(tbu)-OH:
   The same steps as for the grafting of lysine/aspartic acid are reproduced for serine.
4. Addition of the lauroyl fragment on the amine of the serine
   Deprotection of the serine with piperidine (20% in DMF) in order to remove the Fmoc group;
   3 series of washes with DMF;
   addition of lauroyl chloride in the presence of pyridine, addition followed by ninhydrin test (conventional in the art) until complete conversion;
   3 series of washes with DMF;
5. Cleavage of the peptide from the resin and deprotection
   treatment of the resin with a 95/2.5/2.5 TFA/TIS/H₂O mixture
   3 precipitations with ether, centrifugation.
   Solution is made with a part of the pellet for analysis.

The post-synthesis analysis is carried out as follows:
The analysis is carried out on a Waters Acquity H Class UPLC and on a 150×2.1 mm BEH C18 column:
   0 to 100% of acetonitrile in 8.5 min, then 100% to 0% of acetonitrile in 2 min, flow rate: 0.6 ml/min.

The mass spectrometry analysis is carried out on a Waters LC/MS system composed of 2695 separation module alliance; X-Bridge C18 column, 3.5 p.m, 4.6 x 150 mm; 2996 photodiode array detector; SQ mass detector 2 (analyzer: quadripole and source : ES+), flow rate: 0.6 ml/min:
3 to 97% of acetonitrile in 15 min,
then a plateau at 97% of acetonitrile for 2 min,
then 97% to 3% of acetonitrile in 2 min, then a plateau at 3% of acetonitrile for 2 min.

The purification is carried out as follows:
The purification is carried out on a Waters LC4000 preparative HPLC and on a Vydac denali column 50×300 mm, 10 micron, in an H₂O (0.1% TFA)/acetonitrile (0.1% TFA) mixture.

The post-purification analysis is carried out as follows:
Analysis of the fractions collected on HPLC according to the protocol described above. The fractions corresponding to the specifications are combined, freeze-dried, and then re-analyzed by HPLC and by mass spectrometry according to the protocols described above.
The final product essentially consists of the lauroyl-SDKP-OH peptide, at more than 90%.
The impurities present are deletion peptides such as Ac-P-OH, Ac-KP-OH or Ac-DKP-OH.
The solvents, activators, protections, . . . are eliminated as the synthesis proceeds, during the precipitation with ether (volatile) and then during the purification and, finally, the freeze-drying.

### Example 2: Neogoutte Preparation

A neogoutte according to the present invention can be prepared according to the procedure disclosed in FR 2 991 196 on the basis of the amounts according to Table 1 below:

TABLE 1
Compounds involved in the preparation of the neogoutte:

| | | | | | Weight % |
|---|---|---|---|---|---|
| | | Trade | | Weight | in the |
| | Compound | name | Supplier | (mg) | method |
| Aqueous | Water | — | — | 1500 | 75.00 |
| phase | PEG 40 | Myrj s40 | Croda | 215 | 10.75 |
| | stearate | | | | |
| Lipid | Phospho- | Phospho- | Lipoid | 45 | 2.25 |
| phase | lipids | lipon | | | |
| | Olive oil | | | 115 | 5.75 |
| | Wax | Lipocire | Gattefossé | 115 | 5.75 |
| | Palmitoyl- | — | Creative | 9.99 | 0.4995 |
| | KTTKS | | Peptide | | |
| | 1% solution | | Gen Pep | 0.01 | 0.0005 |
| | Lauroyl-SDKP- | | | | |
| | OH | | | | |

N.B.: The same compounds as those described in FR 2 991 196 were used in the context of the present invention.

It has also been noticed possibilities for modifications of this formulation during the method for producing the neogouttes which is described below:
the amount of water can be from 60% to 90% by weight relative to the total weight of the compounds involved, for example 77.2%;
the amount of PEG 40 stearate can be from 5% to 20% by weight relative to the total weight of the compounds involved, for example 9.2%;
the amount of phospholipids can be between 1.5% and 3% by weight relative to the total weight of the compounds involved, for example 1.75%;
the amount of olive oil can be between 3% and 10% by weight relative to the total weight of the compounds involved, for example 4.49%;
the amount of wax can be between 3% and 10% by weight relative to the total weight of the compounds involved, preferably of the same weight as the olive oil, for example 4.49%;
the amount of palmitoyl-KTTKS can for example be between 0.01% and 10% by weight relative to the total weight of the compounds involved, for example 0.05%;
the amount of lauroyl-SDKP-OH can for example be between 0.00001% and 1% by weight relative to the total weight of the compounds involved, for example 0.00006%.
   A. Preparation of the aqueous phase

The aqueous phase was prepared by dissolving the Myrj S40 surfactant, dissolved in 1× phosphate buffered saline (PBS), in water.

### B. Preparation of the Lipid Phase 30

The lipid phase was prepared by mixing soya bean oil (Soybean oil, Sigma Aldrich), paraffin (semi-synthetic glycerides, Suppocire NC, Gattefossé, France), soybean phospholipids (Phospholipon 75, Lipoid, Germany) and 0.1% by weight of Dil fluorophore (1,1′-dioctadecyl-3,3,3′,3′-tetramethylindocarbocyanine perchlorate, Sigma Aldrich). The lipid phase thus prepared contains 16% by weight of phospholipids and 84% by weight of lipids.

C. Preparation of the neogouttes by ultrasonication

20% of the lipid phase were dispersed in 80% of the aqueous phase, resulting in a mixture having a phospholipids/Myrj S40 ratio of 0.18 and a Myrj S40/(oil+wax) ratio of 0.55.

The emulsification of the mixture was then carried out with a 3 mm ultrasonic probe, according to (10 s ON/30 s OFF) sonication cycles for 10 min.

The nanoemulsion suspensions can then be dialyzed against 500 ml of 1× PBS overnight. They were then recovered, diluted to a content of 10% by weight, filtered through pores of 0.2 pm and, finally, stored at 4° C. until use.

### Example 3: Dilution

It is possible to dilute neogouttes according to the present invention.

A hydrophilic solvent can be used for this purpose. Water is typically used. It is also possible to add preservatives, antimicrobial agents, or any other compound (e.g. excipient) commonly used in cosmetic, dermocosmetic and/or pharmaceutical formulations.

From a practical point of view, the neogouttes are diluted simply by adding a diluting solvent (e.g. water) to the concentrated formulation of neogoutte (as produced in example 2 above). The addition of the diluting solvent can be carried out with stirring (e.g. by a turbine), or the stirring can be carried out after the addition of the diluting solvent.

Thus, all the neogouttes presently described can be diluted in aqueous solutions, for example to ⅕^{th}, 1/10^{th}, 1/20^{th} or else 1/50^{th} (see procedure described in detail below).

For example, the neogouttes of example 2 can be diluted to 1/10^{th} in an aqueous solution. An aqueous solution is then added with stirring to the formulation obtained according to example 2 until the initial solution is diluted to 1/10^{th} (i.e. 9 volumes of aqueous diluting solution added to 1 volume of the formulation according to example 2).

### Example 4: Comparative tests

In the context of the present invention, a comparative clinical test between neogouttes without lauroyl-SDKP-OH and neogouttes with lauroyl-SDKP-OH were carried out on healthy volunteers.

In the case of the formulations comprising the neogouttes without lauroyl-SDKP-OH, the missing portion of lauroyl-SDKP-OH in the neogouttes was filled with “Myrj S40”.

The amount of lauroyl SDKP-OH dosed into the neogouttes in the final product used in the context of these experiments was less than 1×10⁻⁵% by weight relative to the total weight of formulation.

Nevertheless, even at these levels of concentration, physiological effects were reported.

1) Smoothing effect
A significant smoothing effect was measured compared with placebo in 75% of cases as early as 14 days with an average roughness decreased by 11%.
Variations up to −19%

2) Anti-wrinkle effect
A significant anti-wrinkle effect was measured compared with placebo in 81% of cases as early as 14 days and 77% at D28 with a variation in the relief of −14% and −16%, respectively.
Variations up to −59%

3) Restructuring/anti-aging effect
A significant restructuring/anti-aging effect was measured in 81% of cases on D14 and 69% on D28 with an increase in the isotropy of 12% and 14%, respectively.
Variations up to +28%. These results are summarized in FIG. 1.

4) Redensifying effect
A redensifying effect was measured in 68% of cases on D14 and 90% on D28, with an average increase in the skin density of respectively 12% and 37%. These results are summarized in FIG. 2.

5) Effect on the dermal thickness
A significant increase in the thickness of the dermis was measured in 72% of patients.

6) Moisturizing effect
A significant moisturizing effect was measured compared with placebo.
Variations up to +20%

7) Tonifying effect
A significant improvement in the tonicity of the skin was measured in 53% of subjects. These results are summarized in FIG. 3.

These particularly encouraging results show the advantage of the choice of the technology according to patent FR 2 991 196 A applied to a modified formula of SDKP according to the present invention.

1. A peptide comprising a peptide strand of formula SDKP, or a biological peptide analog thereof, wherein at least one linear or branched, saturated or unsaturated, C₁₃-C₅₀ fatty chain is grafted at the N- and/or C-terminal end of the peptide strand.

2. The peptide conjugate as claimed in claim 1, wherein the biological analog comprises a peptide strand of formula (II) below:
—Y₁—Y₂—Y₃—Y₄— (II)
wherein,
Y₁ represents the N-terminal end of the peptide strand (II),
the Y₃—Y₄ or —Y₄ fragments possibly being absent from the peptide strand of formula (II),
Y₁ represents serine or a structural analog of serine,
Y₂ represents aspartic acid or a structural analog of aspartic acid,
Y₃ represents lysine or a structural analog of lysine, and
Y₄ represents proline or a structural analog of proline.

3. The peptide conjugate or biological analog thereof as claimed in claim 1, wherein said conjugate is of formula (I) below:
R₁—X₁—X₂—X₃—X₄—A₁—R₂ (I)
wherein:
R_{1,} which is on the N-terminal side, is a hydrogen atom, or a fatty chain chosen from a substituted or unsubstituted, linear or branched C₁₃-C₅₀ alkyl group, a substituted or unsubstituted, linear or branched C₂₁-C₅₀ alkylaryl group, a substituted or unsubstituted, linear or branched C₁₃-C₅₀ alkenyl group, or an R₃—CO—, R₃—OCO— or R₃—COO— group wherein R₃ is a substituted or unsubstituted, linear or branched C₁₃-C₄₉ alkyl or alkenyl group, the substitutions including F, Cl, or any lipophilic heteroatom or heteroatom group;
X₁ is an (L)-serine condensate,
X₂ is an (L)- and/or (D)-aspartic acid or (L)- and/or (D)-glutamic acid condensate,
X₃ is an (L)- and/or (D)-lysine, (L)- and/or (D)-arginine or (L)- and/or (D)-ornithine condensate,
X₄ is an (L)- and/or (D)-proline condensate,
X_{4,} or X₃ and X_{4,} optionally being absent,
the bonds linking X₁ to X₂, X₂ to X₃ where appropriate, and X₃ to X₄ where appropriate, possibly being peptide or pseudopeptide bonds,
A₁ is a covalent bond, an NH group or an oxygen atom,
R₂ is a hydrogen atom, or a fatty chain chosen from a substituted or unsubstituted, linear or branched C₁₃-C₅₀ alkyl group, a substituted or unsubstituted, linear or branched C₂₁-C₅₀ alkylaryl group, a substituted or unsubstituted, linear or branched C₁₃-C₅₀ alkenyl group, or an R₄—CO— or R₄—OCO— group wherein R₄ is a substituted or unsubstituted, linear or branched C₁₃-C₅₀ alkyl or alkenyl group, the substitutions including F, Cl, or any lipophilic heteroatom or heteroatom group,
R₁ and R₂ are not hydrogen atoms, or a pharmaceutically acceptable salt thereof.

4. A lipid nanoparticle comprising:
a core consisting of a lipid phase (L₁);
at least one surfactant comprising a hydrophilic portion and a lipophilic portion;
an internal membrane surrounding said core consisting of a lipid phase (L₂), comprising the lipophilic portion of said surfactant;
an external membrane, surrounding said internal membrane, consisting of an aqueous phase (A₁) comprising the hydrophilic portion of said surfactant; and
at least one peptide conjugate, comprising a peptide strand of formula SDKP, or a biological peptide analog thereof, wherein at least one linear or branched, saturated or unsaturated C₃-C₅₀ fatty chain is grafted at the N- and/or C-terminal end of the peptide strand, and said peptide conjugate is such that its lipophilic portion is in the lipid phase L₂ and its hydrophilic portion is in the phase A₁.

5. The lipid nanoparticle as claimed in claim 4, wherein the peptide conjugate or the biological analog thereof is a peptide conjugate comprising a peptide strand of formula SDKP, or a biological peptide analog thereof, wherein at least one linear or branched, saturated or unsaturated C₁₃-C₅₀ fatty chain is grafted at the N- and/or C-terminal end of the peptide strand.

6. The lipid nanoparticle as claimed in claim 4 further comprising the peptide conjugate of formula Ac-SDKP-OH, Ac-SDKP-NH₂ or a mixture thereof.

7. The lipid nanoparticle as claimed claim 4, wherein the peptide conjugate is of formula (I):
R₁—X₁—X₂—X₃—X₄—A₁—R₂ (I)
wherein:
R₁, which is on the N-terminal side, is a hydrogen atom, a substituted or unsubstituted, linear or branched C₃-C₅₀ alkyl group, a substituted or unsubstituted, linear or branched C₈-C₅₀ alkylaryl group, a substituted or unsubstituted, linear or branched C₃-C₅₀ alkenyl group, or an R₃—CO—, R₃—OCO— or R₃—COO— group wherein R₃ is a substituted or unsubstituted, linear or branched C₃-C₅₀ alkyl or alkenyl group, the substitutions including F, Cl, or any lipophilic heteroatom or heteroatom group, and
X₁ is an (L)-serine condensate,
X₂ is an (L)- and/or (D)-aspartic acid or (L)- and/or (D)-glutamic acid condensate,
X₃ is an (L)- and/or (D)-lysine, (L)- and/or (D)-arginine or (L)- and/or (D)-ornithine condensate,
X₄ is an (L)- and/or (D)-proline condensate,
X₄, or X₃ and X₄ optionally being absent,
the bonds linking X₁ to X₂, X₂ to X₃ where appropriate, and X₃ to X₄ where appropriate, being peptide or pseudopeptide bonds,
A₁ is a covalent bond, an NH group or an oxygen atom,
R₂ is a hydrogen atom, a substituted or unsubstituted, linear or branched C₃-C₅₀ alkyl group, a substituted or unsubstituted, linear or branched C₈-C₅₀ alkylaryl group, a substituted or unsubstituted, linear or branched C₃—C₅₀ alkenyl group, or an R₄-CO— or R₄—OCO— group wherein R₄ is a substituted or unsubstituted, linear or branched C₃-C₅₀ alkyl or alkenyl group, the substitutions including F, Cl, or any lipophilic heteroatom or heteroatom group
R₁ and R₂ not being able to both be hydrogen atoms,
or a pharmaceutically acceptable salt thereof.

8. The lipid nanoparticle as claimed in claim 4, wherein:
R₁ is a substituted or unsubstituted, linear or branched C₆-C₃₀ alkyl group or an R₃—CO—group,
X₁ is an (L)-serine condensate,
X₂ is an (L)- and/or (D)-aspartic acid or (L)- and/or (D)-glutamic acid condensate,
X₃ is an (L)- and/or (D)-lysine, (L)- and/or (D)-arginine or (L)- and/or (D)-ornithine condensate,
X₄ is an (L)- and/or (D)-proline condensate,
X₄ or X₃ and X₄, optionally being absent,
the bonds linking X₁ to X₂, X₂ to X₃ where appropriate, and X₃ to X₄ where appropriate, possibly being peptide or pseudopeptide bonds,
A₁ is a covalent bond, an NH group or an oxygen atom, R₂ is a substituted or unsubstituted, linear or branched C₅-C₃₀alkyl or alkenyl group, the substitutions including F, Cl, or any lipophilic heteroatom or heteroatom group such as a guanidine or guanidinium group or a pharmaceutically acceptable salt thereof.

9. The lipid nanoparticle as claimed in claim 4, wherein said lipid nanoparticle comprises at least one of the following constituents:
at least one C₅-C₃₀ fatty acid, which is optionally hydrogenated and/or in glycol ester form, in the (L₁) phase,
a C₁-C₃₀ fatty acid ester of polyoxyethylene (10-100) as surfactant,
at least one C₅-C₃₀ fatty acid, which is optionally hydrogenated and/or in glycol ester form, in the (L₂) phase, and/or
at least one preservative in the (Ai) phase.

10. The lipid nanoparticle as claimed in claim 4, wherein the peptide conjugate comprises the sequence (L)S-(L)D-(L)K-(L)P.

11. A method for producing a lipid nanoparticle as claimed in claim 4, wherein said method comprises the following steps:
a. preparing a lipid phase and an aqueous phase, at least one of the two phases comprising a surfactant, at least one of the two phases comprising the peptide strand of formula SDKP, or a biological peptide analog thereof, grafted with a fatty chain chosen from a substituted or unsubstituted, linear or branched C₁₃-C₅₀ alkyl group, a substituted or unsubstituted, linear or branched C21-C₅₀ alkylaryl group, a substituted or unsubstituted, linear or branched C13-C₅₀ alkenyl group, or an R₃—CO—, R₃—OCO— or R₃—COO—group wherein R₃ is a substituted or unsubstituted, linear or branched C₁₃-C₄₉ alkyl or alkenyl group or an R₄—CO—or R₄—OCO— group wherein R₄ is a substituted or unsubstituted, linear or branched C13-C₅₀ alkyl or alkenyl group, the substitutions including F, Cl, or any lipophilic heteroatom or heteroatom group;
b. emulsifying said lipid phase and said aqueous phase, resulting in the formation of lipid nanoparticles, optionally under pressure;
c. recovering the lipid nanoparticles formed.

12. A cosmetic formulation comprising one or more lipid nanoparticles as claimed in claim 4.

13. The cosmetic formulation as claimed in claim 12, comprising between 1% and 25% by weight of nanoparticles comprising:
a core consisting of a lipid phase (L₁);
at least one surfactant comprising a hydrophilic portion and a lipophilic portion;
an internal membrane surrounding said core consisting of a lipid phase (L₂), comprising the lipophilic portion of said surfactant;
an external membrane, surrounding said internal membrane, consisting of an aqueous phase (A₁) comprising the hydrophilic portion of said surfactant; and
at least one peptide conjugate, comprising a peptide strand of formula SDKP, or a biological peptide analog thereof, wherein at least one linear or branched, saturated or unsaturated C₃-C₅₀ fatty chain is grafted at the N- and/or C-terminal end of the peptide strand, and said peptide conjugate is such that its lipophilic portion is in the lipid phase L2 and its hydrophilic portion is in the phase Ai.

14. A nanoemulsion comprising:
at least one dispersed lipid phase (L₃), and
at least one continuous aqueous phase (A₂) wherein the aqueous phase comprises at least one surfactant, wherein,
at the interface between the aqueous phase (A₂) and the lipid phase (L₁), there is at least one peptide conjugate comprising a peptide strand of formula SDKP, or a biological peptide analog thereof, wherein at least one linear or branched, saturated or unsaturated, C₁₃-C₅₀ fatty chain is grafted at the N- and/or C-terminal end of the peptide strand and/or in that said nanoemulsion comprises at least one lipid nanoparticle comprising
a core consisting of a lipid phase (L₁);
at least one surfactant comprising a hydrophilic portion and a lipophilic portion;
an internal membrane surrounding said core consisting of a lipid phase (L₂), comprising the lipophilic portion of said surfactant;
an external membrane, surrounding said internal membrane, consisting of an aqueous phase (A₁) comprising the hydrophilic portion of said surfactant; and
at least one peptide conjugate, comprising a peptide strand of formula SDKP, or a biological peptide analog thereof, wherein at least one linear or branched, saturated or unsaturated C₃-C₅₀ fatty chain is grafted at the N- and/or C-terminal end of the peptide strand, and said peptide conjugate is such that its lipophilic portion is in the lipid phase L₂ and its hydrophilic portion is in the phase A₁ wherein optionally the lipids of (L₁) and (L₃), or (L₂) and (L₃) are identical.

15. A method for restricting or preserving the appearance of the skin of a human subject comprising a step of applying on the skin a peptide conjugate comprising a peptide strand of formula SDKP, or a biological peptide analog thereof, wherein at least one linear or branched, saturated or unsaturated, C₁₃-C₅₀ fatty chain is grafted at the N- and/or C-terminal end of the peptide strand.

16. The method as claimed in claim 15 wherein restricting or preserving the appearance of the skin includes anti-aging the skin.

17. A method for restructuring or preserving the appearance of the skin of a human subject comprising a step of applying on the skin a lipid nanoparticle comprising:
a core consisting of a lipid phase (L₁);
at least one surfactant comprising a hydrophilic portion and a lipophilic portion;
an internal membrane surrounding said core consisting of a lipid phase (L₂), comprising the lipophilic portion of said surfactant;
an external membrane, surrounding said internal membrane, consisting of an aqueous phase (A₁) comprising the hydrophilic portion of said surfactant; and
at least one peptide conjugate, comprising a peptide strand of formula SDKP, or a biological peptide analog thereof, wherein at least one linear or branched, saturated or unsaturated C₃-C₅₀ fatty chain is grafted at the N- and/or C-terminal end of the peptide strand, and said peptide conjugate is such that its lipophilic portion is in the lipid phase L₂ and its hydrophilic portion is in the phase A₁.

18. A method for restructuring or preserving the appearance of the skin of a human subject comprising a step of applying on the skin a nanoemulsion comprising:
at least one dispersed lipid phase (L₃), and
at least one continuous aqueous phase (A₂) wherein the aqueous phase comprises at least one surfactant, wherein, at the interface between the aqueous phase (A₂) and the lipid phase (L₁), there is at least one peptide conjugate comprising a peptide strand of formula SDKP, or a biological peptide analog thereof, wherein at least one linear or branched, saturated or unsaturated, C₁₃-C₅₀ fatty chain is grafted at the N- and/or C-terminal end of the peptide strand and/or in that said nanoemulsion comprises at least one lipid nanoparticle comprising:
a core consisting of a lipid phase (Li);
at least one surfactant comprising a hydrophilic portion and a lipophilic portion;
an internal membrane surrounding said core consisting of a lipid phase (L₂), comprising the lipophilic portion of said surfactant;
an external membrane, surrounding said internal membrane, consisting of an aqueous phase (A₁) comprising the hydrophilic portion of said surfactant;
and at least one peptide conjugate, comprising a peptide strand of formula SDKP, or a biological peptide analog thereof, wherein at least one linear or branched, saturated or unsaturated C3-C₅₀ fatty chain is grafted at the N-and/or C-terminal end of the peptide strand, and said peptide conjugate is such that its lipophilic portion is in the lipid phase L₂ and its hydrophilic portion is in the phase A₁. wherein optionally the lipids of (L₁) and (L₃), or (L₂) and (L₃) are identical.

19. A method for restructuring or preserving the appearance of the skin of a human subject comprising a step of applying on the skin a cosmetic formulation comprising one or more lipid nanoparticles comprising:
a core consisting of a lipid phase (L₁);
at least one surfactant comprising a hydrophilic portion and a lipophilic portion;
an internal membrane surrounding said core consisting of a lipid phase (L₂), comprising the lipophilic portion of said surfactant;
an external membrane, surrounding said internal membrane, consisting of an aqueous phase (A₁) comprising the hydrophilic portion of said surfactant; and
at least one peptide conjugate comprising a peptide strand of formula SDKP, or a biological peptide analog thereof, wherein at least one linear or branched, saturated or unsaturated C₃-C₅₀ fatty chain is grafted at the N- and/or C-terminal end of the peptide strand, and said peptide conjugate is such that its lipophilic portion is in the lipid phase L₂ and its hydrophilic portion is in the phase A₁.


SEQUENCE LISTING (9 sequences)

SEQ ID NO: 1 - protein, 4 aa, Artificial Sequence
  SYNTHETIC
SDKP

SEQ ID NO: 2 - protein, 4 aa, Artificial Sequence
  SYNTHETIC
  MISC_FEATURE: N-TERMINAL ACETYLATION
SDKP

SEQ ID NO: 3 - protein, 4 aa, Artificial Sequence
  SYNTHETIC
  MISC_FEATURE: N-TERMINAL ACETYLATION AND C-TERMINAL AMIDATION
SDKP

SEQ ID NO: 4 - protein, 5 aa, Artificial Sequence
  SYNTHETIC
  MISC_FEATURE (1)..(1): Xaa can be any naturally occurring amino acid
XSDKP

SEQ ID NO: 5 - protein, 5 aa, Artificial Sequence
  SYNTHETIC
  MISC_FEATURE (1)..(1): Palmitoyl is attached to position 1
KTTKS

SEQ ID NO: 6 - protein, 4 aa, Artificial Sequence
  SYNTHETIC
  MISC_FEATURE: N-TERMINAL ACETYLATION
  MISC_FEATURE (1)..(2): S IN POSITION 1 AND D IN POSITION 2 ARE BONDED BY A PSEUDOPEPTIDE BOND [CH2-NH]
SDKP

SEQ ID NO: 7 - protein, 4 aa, Artificial Sequence
  SYNTHETIC
  MISC_FEATURE: N-TERMINAL ACETYLATION
  MISC_FEATURE (2)..(3): D IN POSITION 2 AND K IN POSITION 3 ARE BONDED BY A PSEUDOPEPTIDE BOND [CH2-NH]
SDKP

SEQ ID NO: 8 - protein, 4 aa, Artificial Sequence
  SYNTHETIC
  MISC_FEATURE: N-TERMINAL ACETYLATION
  MISC_FEATURE (3)..(4): K IN POSITION 3 AND P IN POSITION 4 ARE BONDED BY A PSEUDOPEPTIDE BOND [CH2-NH]
SDKP

SEQ ID NO: 9 - protein, 4 aa, Artificial Sequence
  SYNTHETIC
  MISC_FEATURE (1)..(1): H atom, C13-C50 alkyl, C21-C50 arylalkyl, R3-CO- or R3-OCO-, wherein R3 is a C13-C49 alkyl or alkenylis, is attached to position 1.
  MISC_FEATURE (2)..(2): Xaa in position 2 is D or N
  MISC_FEATURE (3)..(3): Xaa in position 3 is K, R or ornithine
  MISC_FEATURE (4)..(4): A bond, NH or O is attached to position 4, which creates position 5, and an H atom, C13-C50 alkyl, C21-C50 arylalkyl, R4-CO- or R4-OCO-, wherein R4 is a C13-C50 alkyl or alkenyl, is attached to position 5.
SXXP